(19) United States
(12) Patent Application Publication
(10) Pub. No.: US 2004/0073212 A1
(43) Pub. Date: Apr. 15, 2004

(54) Extracorporeal fixing device for a bone fracture

(76) Inventor: Jung Jae Kim, Seoul (KR)

Correspondence Address: Ladas & Parry, 26 West 61st Street, New York, NY, 10023, US

(21) Appl. No.: 10/395,938

(22) Filed: Mar. 24, 2003

(30) Foreign Application Priority Data
Oct. 15, 2002 (KR) 10-2002-62822

(51) Int. Cl. A61B 17/58

(52) U.S. Cl. 606/56

(57) ABSTRACT

The present invention relates to extracorporeal fixing device for a bone fracture and soft tissue trauma caused by bone fracture, and in particular, to the device which can be easily and versatilely installed depending on the condition of fractured bone and destructed soft tissue and allows partial modification without dismantling the entire structure and also keep stable fixating condition at the same time.

The present invention discloses an extracorporeal fixing device for a bone fracture including a pair of ring members through which an object bone structure pass in a longitudinal direction of the object bone structure and are spaced apart by a certain distance, a rod which is installed between the ring members and is adapted to setting a position of each member, a pair of ring-rod fixing clamps which are engaged on the certain position of each ring member and rotatably fix both ends of the rod with respect to each ring member, a fixing pin which is movably engaged to the rod and fixes a fracture portion, and a rod-pin fixing clamp which movably fixes the fixing pin to the rod.

### BACKGROUND OF THE INVENTION

1. Field of the Invention

The present invention relates to an extracorporeal fixing device for a bone fracture and soft tissue trauma caused by bone fracture, and in particular, to the device which can be easily and versatilely installed depending on the condition of the fractured bone and destructed soft tissue and allows partial modification without dismantling the entire structure.

2. Description of Prior Art

External fixation devices for bone fracture can be classified into 3 groups which are simple type, clamp type and ring type, respectively.

The simple type external fixator, each clamp engaging single fixation pin to the external frame of interconnected rod, can be installed versatilely depending on the condition of soft tissue, but it is difficult to be installed on the fractured limb. The demerit of the simple external fixator is that the fixated region lacks stability because of its unilateral fixation. In particular, when the fractured bone segments are short, the distance between the fixation pins become too short to assure adequate stability.

The clamp type external fixator, each clamp engaging a plurality of fixation pins to the external frame of interconnected rod, doesn't allow the directional modification of the fixation pins. Same as the simple external fixator, its single external frame of interconnected rod can not assure adequate stability.

By contrast, the ring type external fixator, often referred to as the Ilizarov External Fixator, is the most stable external fixator. The development of the Ilizarov external fixator motivated the organization of the distraction osteogenesis. The distraction Osteogenesis, an epochal method in orthopedic surgery, resolves many deformities which seemed inoperable before. That is, extremity length discrepancies resulted from extensive range bone defects caused by trauma or infection and from congenital pathologies were inoperable or difficult to correct before, the distraction osteogenesis can solve those problem.

Conventional Ilizarov external fixator as shown in FIG. 1 includes three basic elements: a plurality of ring members(1) disposed coaxially about the bone segments to be fixated; transverse wires(2) for fixating the bone segments to the ring members; and 3 or 4 interconnected rods(3) being engaged to the ring members(1) at regular intervals of 90° or 120°. At this occasion, continuous radiation is required during the installation to monitor the position of each transverse wire(2) so that the each wire is disposed in the accurate site of bone segments to be fixated.

Above mentioned Ilizarov External Fixator, though widely used as a unique method of correcting the limb deformities, has problems in treating traumatized bone as follows.

First, due to the regular interval (90° or 120°) and fixed posture of the interconnected rod, it is difficult to obtain sufficient space necessary for treating soft tissue injury accompanied by the bone fracture.

Second, fixating of the fractured bone only with the transverse wires increases the possibility of blood vessel injury and neuro trauma. Also, perforation of muscle can cause pain, discomfort and subsequently joint stiffness which lead to bad prognosis to the patients who should wear the device for a long time.

Third, once the fixator is installed, the ring members cannot be detached from the interconnected rods. Therefore each of the ring members and rods should be disassembled and then reconstructed when additional modification is necessary.

Fourth, there is a risk of overexposure to radiation because image intensifier(C-arm) should be used from start to finish for accurate installation required on account of the difficulties in adjustment after installation.

Finally, since it is incompatible with other fixators, the disassembly and reconstruction of each member are needed for the patients putting on other fixator.

U.S. Pat. No. 5,630,814 describes a similar external bone fixation device with FIG. 1. which also has above mentioned problems; lack of required space for treating the soft tissue injury, complicated disassembly and reconstruction steps for additional modification, difficulties in adjustment after installation and the risk of overexposure to radiation

Therefore, the present invention is designed for the provision of extracorporeal fixing device for a bone fracture which overcomes the above-discussed disadvantages of conventional bone fixation devices and can be easily installed depending on the condition of the fractured bone and destructed soft tissue without regional restriction, also allows multi-directional modification and transformation, easy addition and disassembly and has the firm fixing strength of conventional Ilizarov external fixator

Also, another object of the present invention is the provision of extracorporeal fixing device for a bone fracture which allows tidy treated region and minimizes the behavior restriction of patient by operating optimal treatment using minimal components.

### SUMMARY OF THE INVENTION

An extracorporeal fixing device for a bone fracture according to the present invention includes a pair of ring members through which an object bone structure pass in a longitudinal direction of the object bone structure and are spaced apart by a certain distance, a rod which is installed between the ring members and is adapted to setting a position of each member, a pair of ring-rod fixing clamps which are engaged on the certain position of each ring member and rotatably fix both ends of the rod 20 with respect to each ring member, a fixing pin which is movably engaged to the rod and fixes a fracture portion, and a rod-pin fixing clamp which movably fixes the fixing pin to the rod.

Also, a ring rod fixing clamp according to an embodiment of the present invention includes a ring fixing member which has one side being detachably engaged to the the ring member and the other side having more than at least one engaging hole, a first rod fixing member in which a through hole through which an end of the rod 20 passes through, is formed in a certain portion of the same, and threaded portions are formed in an upper and lower potion, and a lower threaded potion between the threaded portions passes through the engaging hole of the ring fixing member and is engaged by a nut, and a rod fixing piece which is implemented in such a manner that an upper threaded portion of a first rod fixing member passes through in an upper direction, and a front and back portion of the firs rod fixing member is surrounded, and a through hole corresponding to the through hole of the first rod fixing member is formed in a front and back portion contacting with a front and back surface of the first rod fixing member, for thereby fixing the rod to the through hole of the first rod fixing member by downwardly pressurizing the same based on a tightening force of the nut engaged to the upper threaded portion of the first rod fixing member.

In succession, a rod-pin fixing clamp according to an embodiment of the present invention includes a pin fixing bolt which has a through hole through which a fixing pin passes vertically, second and third opposite rod fixing members in which one end of each of the same has a through hole through which a pin fixing bolt passes through in sequence, and an insertion groove into which an end of the rod is inserted, is formed in the opposite other end of the same, and a pin fixing piece which is installed between a head portion of the pin fixing bolt and the second rod fixing member and fixes the fixing pin to the through hole of the pin fixing bolt by pressurizing the fixing pin based on a tightening force of the nut engaged to the threaded portion of the pin fixing bolt, and includes a radial threaded portion being engaged with a radial threaded portion formed in the upper corresponding surface of the second rod fixing member and preventing a relative rotation.

A ring-rod fixing clamp according to another embodiment of the present invention includes a ring fixing member which has one side being detachably engaged to said ring members, a rod-fixing bolt which has a head portion through which an end portion of the rod 20 passes vertically, a connection member in which a through hole through which the other end of the ring fixing member passes horizontally is formed in one side, and upper and lower corresponding surfaces which are extended from one side in the upper and lower directions are formed in the other side, and a through hole through which a rod fixing bolt passes vertically is formed in each corresponding surface, and a rod fixing piece which is installed between a head portion of the rod fixing bolt and the connection member and fixes the rod to the rod fixing bolt by pressurizing an end portion of the rod based on a tightening force of the nut engaged to the threaded portion of the rod fixing bolt which passes through the connection member, and includes a radial threaded portion being engaged with a radial threaded portion formed in the upper corresponding surface of the connection member and preventing a relative rotation.

An extracorporeal fixing device for a bone fracture according to the other embodiment of the present invention further includes an assistant extracorporeal fixing device for a bone fracture which is movably fixed to the rod and fixes the back of hand and the top of foot.

### BRIEF DESCRIPTION OF THE DRAWING

FIG. 1 shows an elevational view of conventional external fixator for a bone fracture illustrated in operative association with a bone.

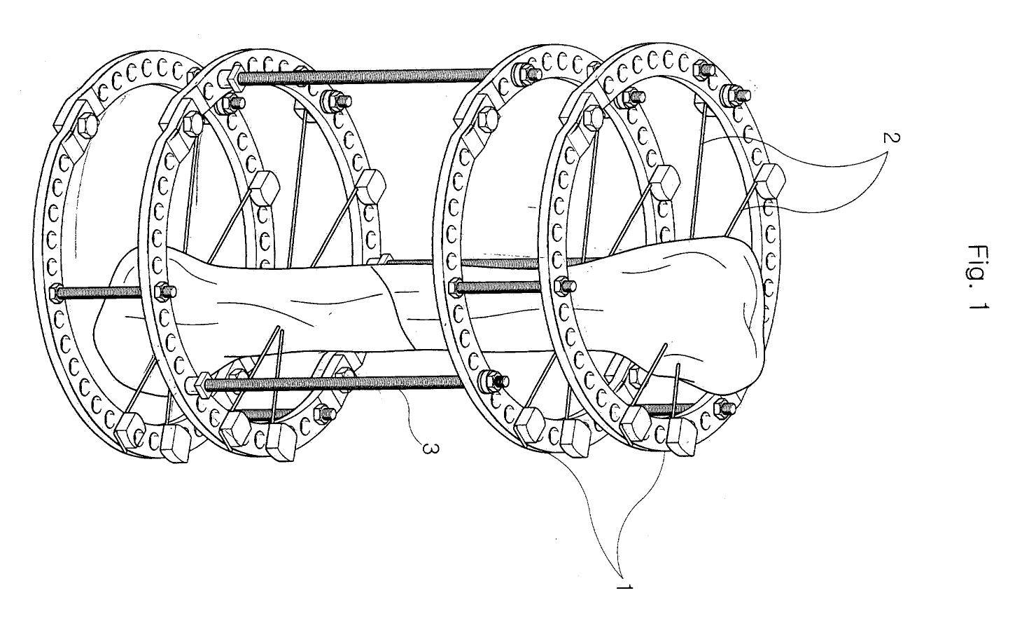

FIG. 2 shows an elevational view of the extracorporeal fixing device for a bone fracture according to the first embodiment of the present invention illustrated in operative association with a bone.

FIG. 3 shows a top view of the versatile circular rings illustrated in the FIG. 2

FIG. 4 shows a perspective view of ring-rod fixing clamp illustrated in the FIG. 2

FIG. 5 shows a sectional view of ring-rod fixing clamp illustrated in the FIG. 2 and FIG. 4

FIG. 6 shows a sectional view of rod-pin fixing clamp illustrated in the FIG. 2

FIG. 7 shows a sectional view of exploded rod-pin fixing clamp illustrated in the FIG. 2 and FIG. 6

FIG. 8 shows a sectional view of rod-pin fixing clamp illustrated in the FIG. 2, FIG. 6 and FIG. 7.

FIG. 9 shows a perspective view of a ring-rod fixing clamp according to the second embodiment of the present invention.

FIG. 10 shows an exploded perspective view of the FIG. 9

FIG. 11 shows an perspective view of a exploded rod-pin fixing clamp according to the third embodiment of the present invention.

FIG. 12 shows an elevational view of the extracorporeal fixing device for a bone fracture according to the fourth embodiment of the present invention illustrated in operative association with a bone.

### DETAILED DESCRIPTION OF THE PREFERRED EMBODIMENT

### First Embodiment

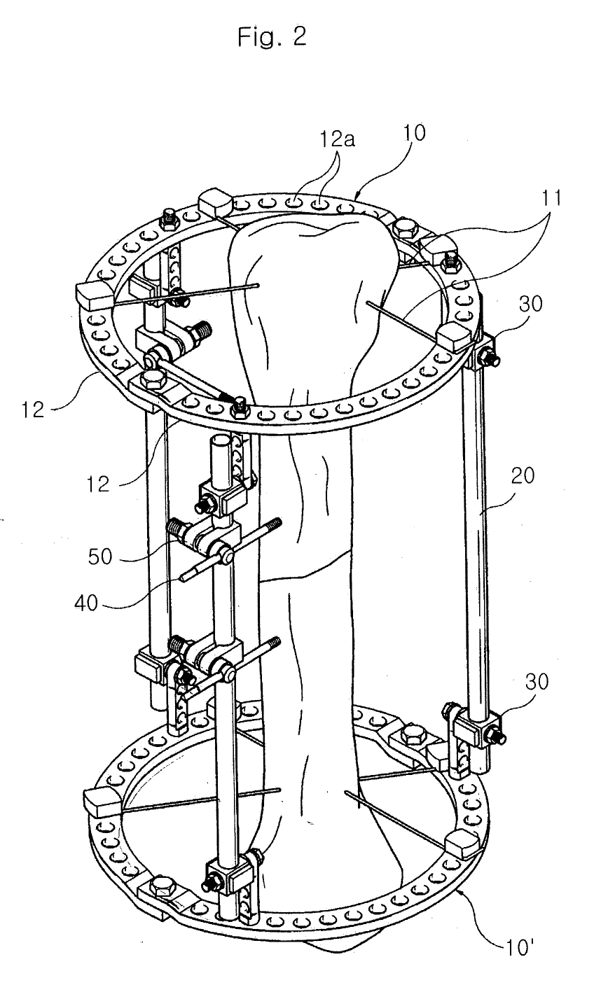

As shown in FIG. 2, an extracorporeal fixing device for a bone fracture according to an embodiment of the present invention includes a pair of distanced ring members 10 and 10, a rod 20 installed between the ring members 10 and 10, a pair of ring-rod fixing clamps 30 which rotatably fix both ends of the rod 20 with respect to each ring member 10, 10, a fixing pin 40 which is movably engaged to the rod 20 and fixes a fracture portion, and a rod-pin fixing clamp 50 which movably fixes the fixing pin 40 to the rod 20.

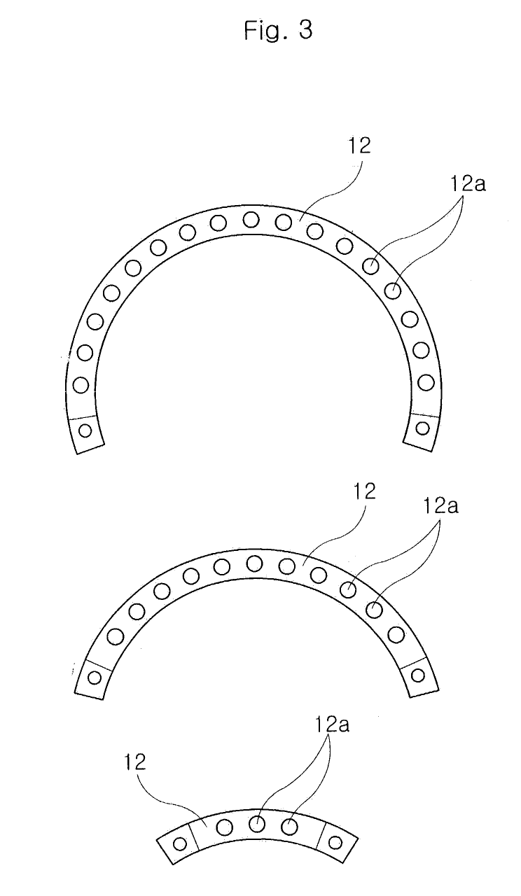

As shown in FIGS. 2 and 3, the ring members 10 and 10 pass through which an object bone structure pass in a longitudinal direction of the object bone structure and are spaced apart by a certain distance and are fixed to the object bone structure by a wire 11. In addition, the ring members 10 and 10 are implemented based on a combination of a plurality of circular rings 12 in which a plurality of engaging holes 12a are formed at a regular interval. Since each circular ring 12 surrounds a calf or femoral region during an operation, the sizes of the same are determined based on a maximum circumference of an operation portion. In general, each ring member 10, 10 is formed based on a combination of a plurality of circular rings. However, in the case of a small bone fracture, one circular ring may be used.

At this time, each ring member 10, 10 is formed of stainless which is not rusted. In another embodiment of the present invention, the ring members 10 and 10 may be formed of a carbon steel which is light and has an excellent radiation permeability.

The rod 20 is installed between the ring members 10 and 10 and is adapted to set a position of each member. The number of the rods 20 may be determined based on a fracture type or may not be used. The fixed position of the same is freely adjusted between the ring members 10 and 10 and are formed of a carbon steel which is light and has an excellent radiation permeability.

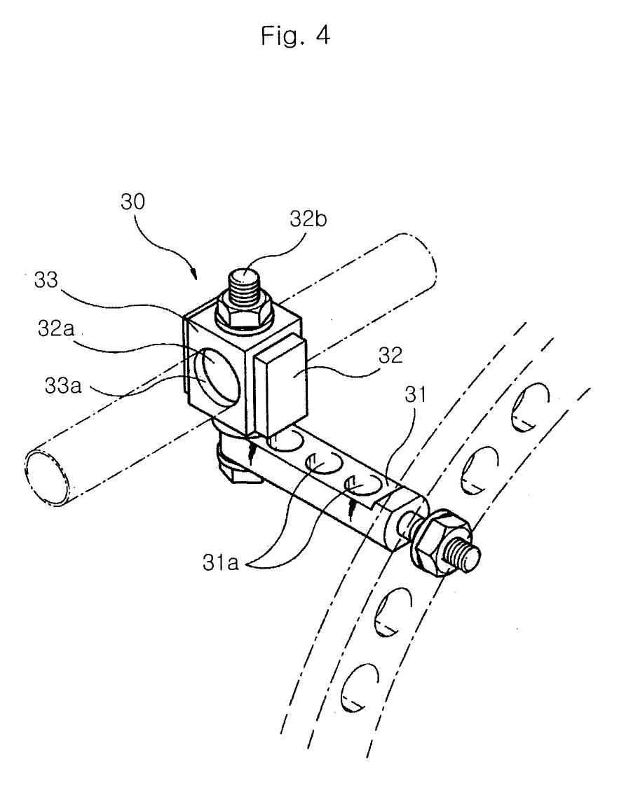

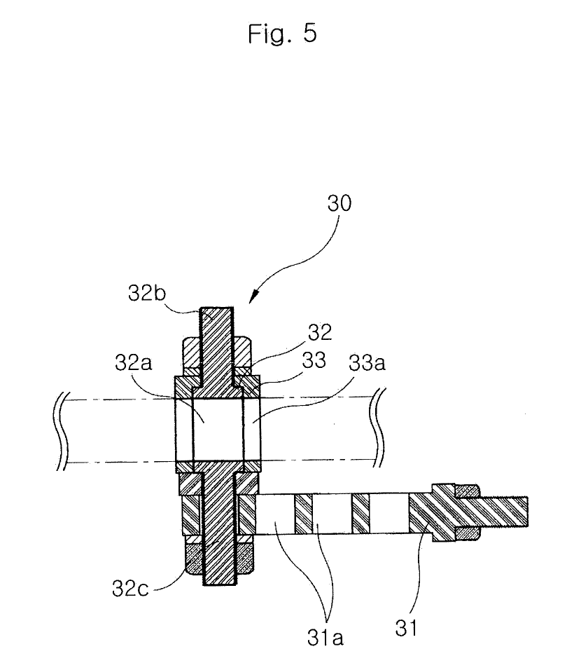

As shown in FIGS. 4 and 5, each ring rod fixing clamp 30 includes a ring fixing member 31 which has one side passing through the engaging hole 12a of the ring member 10, 10 and engaged by a nut and the other side having more than at least one engaging hole 31a, a first rod fixing member 32 in which a through hole 32a through which an end of the rod 20 passes through, is formed in a certain portion of the same, and threaded portions 32b and 32c are formed in an upper and lower potion, and a lower threaded potion 32c between the threaded portions 32b and 32c passes through the engaging hole 31a of the ring fixing member 31 and is engaged by a nut, and a rod fixing piece 33 which is implemented in such a manner that an upper threaded portion 32b of a first rod fixing member 32 passes through in an upper direction, and a front and back portion of the firs rod fixing member 32 is surrounded, and a through hole 33a corresponding to the through hole 32a of the first rod fixing member 32 is formed in a front and back portion contacting with a front and back surface of the first rod fixing member 32, for thereby fixing the rod 20 to the through hole 32a of the first rod fixing member 32 by downwardly pressurizing the same based on a tightening force of the nut engaged to the upper threaded portion 32b of the first rod fixing member 32.

In the thusly constituted ring rod fixing clamp 30, the rod 20 is escaped from the first rod fixing member 32 by removing the tightening force of the nut engaged to the upper threaded portion 32b of the first rod fixing member 32. The first rod fixing member 32 is escaped from the ring fixing member 31 or becomes a relatively rotatable state by removing the tightening force of the nut engaged to the low threaded portion 32c of the first rod fixing member 32. In addition, the ring fixing member 31 is escaped from the ring member 10, 10 or becomes a rotatable state by removing the tightening force of the nut engaged to one side of the ring fixing member 31. Therefore, it is possible to freely change the position of the rod 20 based on the fracture state in a state that the ring member 10, 10 is fixed to an object bone structure.

As shown in FIG. 2, the fixing pin 40 is movably fixed to the rod 20. One end of the same is fixed to the rod-pin fixing clamp 50, and the other end of the same is engaged to the bone structure. At this time, a threaded portion is formed in the other end of the fixing pin 40 for thereby preventing an easier escape from the bone structure. The above threaded portion is fully engaged to the bone structure for preventing a bacteria infection in a skin which may be easily damaged when the threaded portion repeatedly touches the skin because the threaded portion is exposed to the outside. In addition, the threaded portion of the fixing pin 40 has various lengths and diameters which are determined based on a patients age and the size of bone structure. The number of the fixing pin 40 is determined based on the state of fracture and is omitted. The diameter of one end of the same may be the same as the diameter of a thread formed in the other end of the same or may be larger than the same, so that the strength is increased.

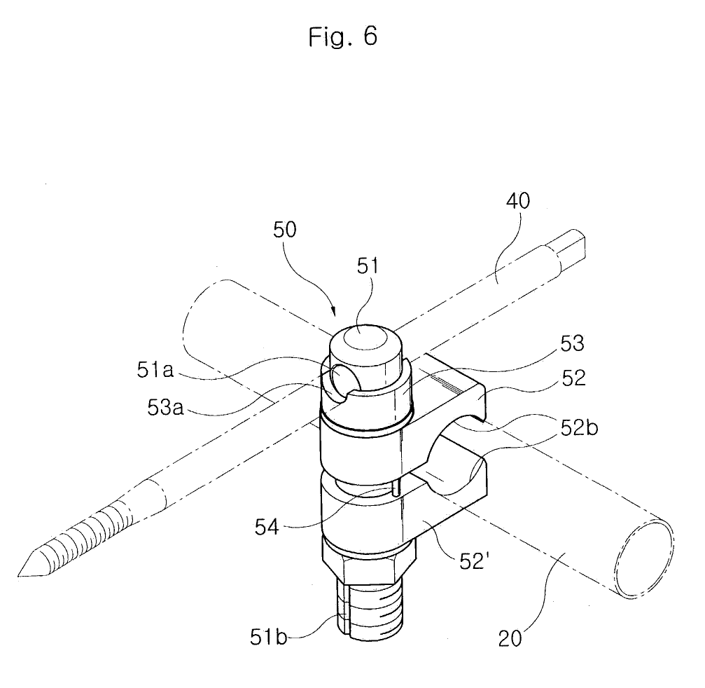

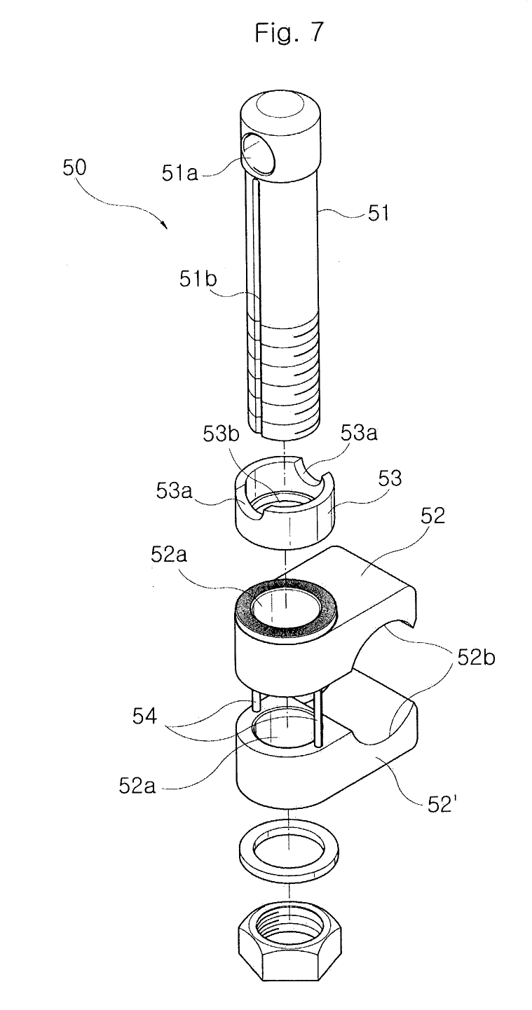

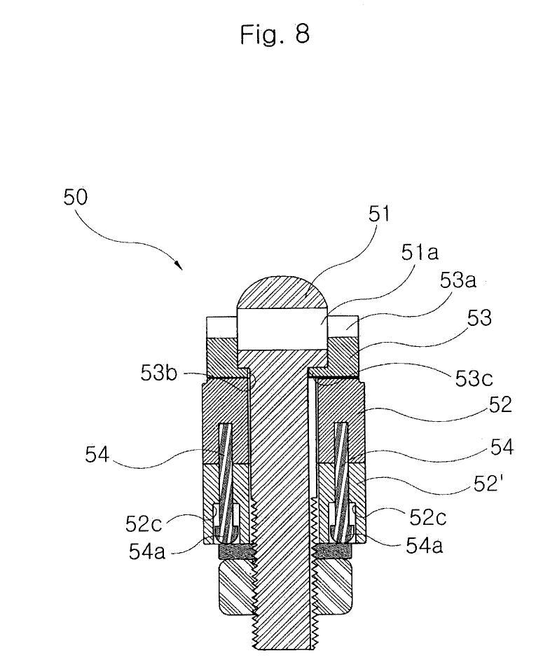

As shown in FIGS. 6 through 8, the rod-pin fixing clamp 50 is adapted to movably fix the fixing pin 40 to the rod 20 and includes a pin fixing bolt 51 which has a through hole 51a through which a fixing pin 40 passes vertically, second and third opposite fixing members 52 and 52 in which one end of each of the same has a through hole 52a through which a threaded portion of a pin fixing bolt 51 passes through in sequence, and an insertion groove 52b into which an end of the rod 20 is inserted, is formed in the opposite other end of the same, a pin fixing piece 53 which is installed between a head portion of the pin fixing bolt 15 and the second rod fixing member 52 and which includes a rod insertion groove 53a into which an end portion of the rod 20 is inserted, for thereby fixing the fixing pin 40 to the through hole 51a of the pin fixing bolt 51 by pressurizing the fixing pin 40 based on a tightening force of the nut engaged to the threaded portion of the pin fixing bolt 51, and a pair of escape prevention rod 54 which has one end passing through a groove 52c formed in both sides of the through hole 52a of the third rod fixing member 52 and being integrally engaged to an opposite surface of the second rod fixing member 52, and the other end in which an engaging portion 54a is formed for preventing an escape of the third rod fixing member 52 in such a manner that the engaging portion 54a is caught by a bottom of the groove 52c formed in both sides of the through hole 52a of the third rod fixing member 52.

At this time, the radial threads are formed in the contact surface contacting with the second rod fixing member 52 and the pin fixing piece 53 and are engaged each other. When the rod 20 is fixed to the through hole 51a of the pin fixing bolt 51, a relative rotation of the second rod fixing member 52 and the pin fixing piece 53 is prevented. In addition, a rotation prevention groove 51b is formed in a threaded portion of the pin fixing bolt 51 in a longitudinal direction. A protrusion 53c which is inserted into the rotation prevention groove 51b is formed in an inner circumference of the through hole 53b of the pin fixing piece 53, so that a relative rotation of the pin fixing piece 51b is prevented with respect to the pin fixing bolt 51. In addition, an escape of the third rod fixing member 52 is prevented by an escape prevention rod 54. Since a pair of escape prevention rods 54 are provided in both sides of the through hole 52a of the third rod fixing member 52, a relative rotation is prevented with respect to the second rod fixing member 52.

Even when there is provided only one escape prevention rod 54, it is possible to prevent a relative escape of the second and third rod members 52 and 52 and to prevent a relative rotation of the pin fixing bolt 51 passing through the through hole 52a of the second and third rod members 52 and 52 and a relative rotation of the second and third rod members 52 and 52.

The operation of an extracorporeal fixing device for a bone fracture according to an embodiment of the present invention will be described.

First, the ring members 10 and 10 pass through an object bone structure and are spaced part at a certain interval and are fixed to an object bone structure, respectively, using multiple wires 11. At this time, the ring members 10 and 10 may be additionally installed in the object bone structure based on the degree of the fracture of the fracture portion.

After the ring members 10 and 10 are fixed to the object bone structure using multiple wires 11, and the ring fixing member 31 of the ring-rod fixing clamp 30 is loosely engaged to the engaging hole 12a of each ring member 10, 10 using a nut. A tightening force of the nut engaged to the upper and lower threaded portions 32b and 32c of the first rod fixing member 32 is weakened, both ends of the rod 20 pass through the rod fixing piece 33 and the first rod fixing member 32. Thereafter, the fracture portion is aligned with the fracture surface using the ring members 10 and 10 fixed to the object bone structure. In the above state, the fracture portion is stably fixed by the ring members 10 and 10 by tightening the nut adapted to fix the ring fixing member 31 to the ring members 10 and 10 and the nut engaged to the threaded portions 32b and 32c of the first rod fixing member 32. At this time, more than rods 20 are preferably provided in order for the ring members 10 and 10 to stably fix the fracture portion.

After the fracture portion of the object bone structure us fixed using the ring members 10 and 10 and the rod 20, the fixing pin 40 is engaged to the fracture portion for thereby obtaining an additional fixed state. In order to engage the threaded portion of the fixing piece 40 to the fracture portion, the rod-pin fixing clamp 50 is loosely installed in the rod 20, and the fixing pin 40 passes through the through hole 51a of the pin fixing bolt 51 for thereby engaging the threaded portion to the fracture portion, so that the fixing pin 40 and the rod-pin fixing clamp 50 are stably fixed by tightening the nut engaged to the threaded portion of the pin fixing bolt 51.

After the fracture portion of the object bone structure is accurately engaged using the ring member 10, 10 the rod 20 and the fixing pin 40, in the case that the fixed position of the rod 20 is changed for an external wound therapy, the fixing pin 40 positioned in the portion of the external wound therapy is removed, and the rod 20 is separated from the first rod fixing member 32 of the ring-rod fixing clamp 30, and the ring fixing member 31 is separated from the ring member 10, 10. Each ring-rod fixing clamp 30 is engaged to another position of each ring member 10, 10, and then the separated rod 20 is fixed to the ring-rod fixing clamp 30. In a state that the position of the ring member 10, 10 is maintained, it is possible to easily perform an external wound therapy by obtaining a space for the external wound therapy by simply changing the positions of the rod 20 and the ring-rod fixing clamp 30 and the fixing pin 40 and the rod-pin fixing clamp 50.

As described above, when the external wound therapy is performed, it is possible to obtain a space for the external wound therapy by simply changing the position of the rod 20 positioned in an external wound portion. In addition, it is possible to obtain an external wound therapy space by fully separating the rod 20 from the ring member 10, 10. At this time, it is possible to maintain a desired stability using more than two rods 20 in each ring member 10, 10.

When a desired stability is obtained based on the above described engagement, the wire 11 which is adapted to fix the ring member 10, 10 to the object bone structure is removed, and the nut engaged to the threaded portions 32b and 32c formed in the upper and lower portions of the first rod fixing member 32 of the ring-rod fixing clamp 30 is loosened for thereby removing the ring member 10, 10 from the rod 20, so that it is possible to implement a desired change based on a simple fixing device.

### Second Embodiment

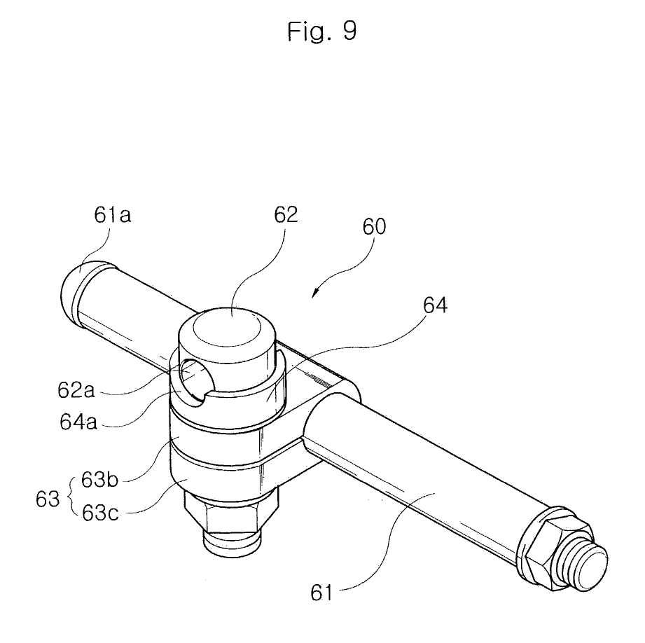

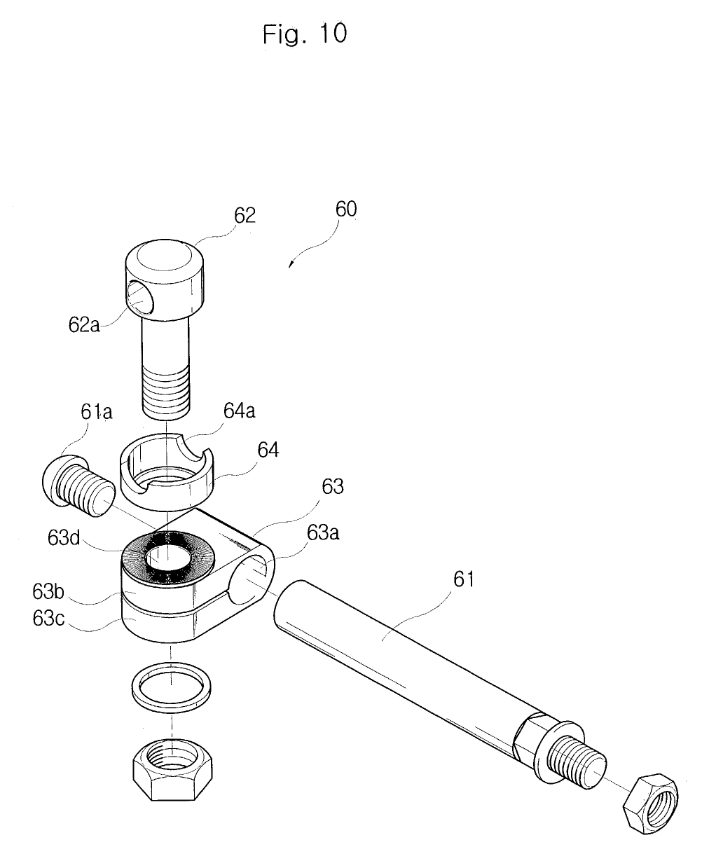

As shown in FIGS. 9 and 10, the ring-rod fixing clamp 60 according to a second embodiment of the present invention includes a ring fixing member 61 which has one end passing through a ring member 10, 10 and being engaged by a nut, a rod-fixing bolt 62 which has a head portion through which an end portion of the rod 20 passes vertically, a connection member 63 in which a through hole 63a through which the other end of the ring fixing member 61 passes horizontally is formed in one side, and upper and lower corresponding surfaces 63b and 63c which are extended from one side in the upper and lower directions are formed in the other side, and a through hole 63d through which a rod fixing bolt 62 passes vertically is formed in each corresponding surface 63b, 63c, and a radial threaded portion is formed in a surrounding portion of the entrance of the through hole 63d of the upper corresponding surface 63b, and a fixing piece 64 which is installed between a head portion of the rod fixing bolt 62 and the connection member 63 and in which a rod insertion groove 64a into which a part of the rod 20 is inserted is formed in one end, for thereby fixing the rod 20 to the through hole 62a of the rod fixing bolt 62 by pressurizing an end portion of the rod 20 based on a tightening force of the nut engaged to the threaded portion of the rod fixing bolt 62 which passes through the connection member 63.

At this time, since a radial threaded portion engaged with a radial threaded portion formed in the upper corresponding surface 63b of the connection member 63 is formed in the other surface of the rod fixing piece 64, in the rod fixing piece 64, a relative rotation is prevented with respect to the upper corresponding surface 63b of the connection member 63.

Since a certain section from the threaded portion formed in one end of the ring fixing member 61 to the other end is formed in a hexagonal shape, the threaded portion passes through the engaging hole 12a of the ring member 10, 10, and when engaging the nut, the hexagonal column section is fixed using a spanner, etc. In a state that the rotation of the ring fixing member 61 is prevented, it is possible to stably engage the ring fixing member 61 to the ring member 10, 10. In addition, an escape prevention cap 61a is detachably engaged to the other end of the ring fixing member 61 for thereby preventing an escape of the connection member 63.

Since the operation of an extracorporeal fixing device for a bone fracture according to a second embodiment of the present invention is the same as the first embodiment of the present invention, the descriptions of the same will be omitted.

### Third Embodiment

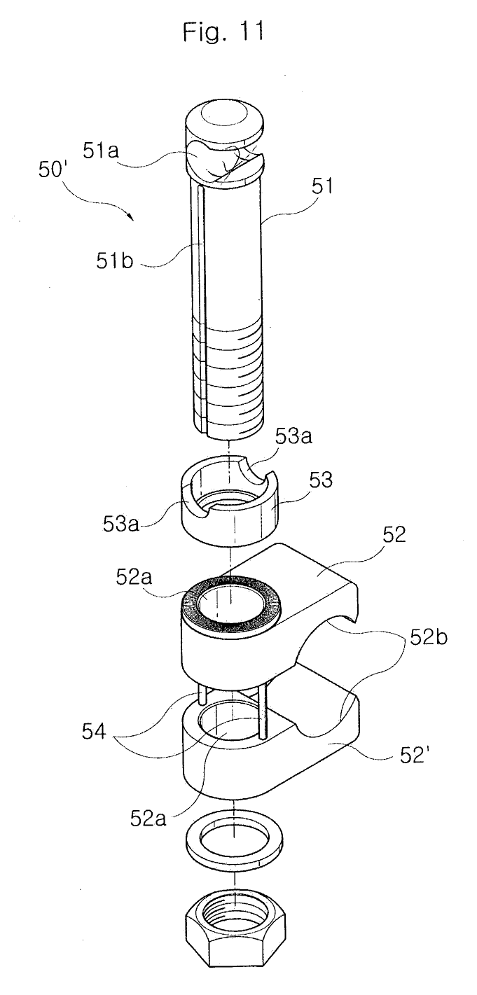

As shown in FIG. 11, according to an extracorporeal fixing device for a bone fracture according to a third embodiment of the present invention, one side of the through hole 51a formed in the pin fixing bolt 51 of the rod-pin fixing clamp 50 is opened, so that the fixing pin 40 is inserted through the opened one side or is escaped therefrom. Therefore, in a state that the rod 20 and the fixing pin 40 are engaged, it is possible to implement an attaching and detaching operation of the rod-pin fixing clamp 50.

Since the operation of the third embodiment of the present invention is the same as the first embodiment of the present invention, the descriptions of the same will be omitted.

### Fourth Embodiment

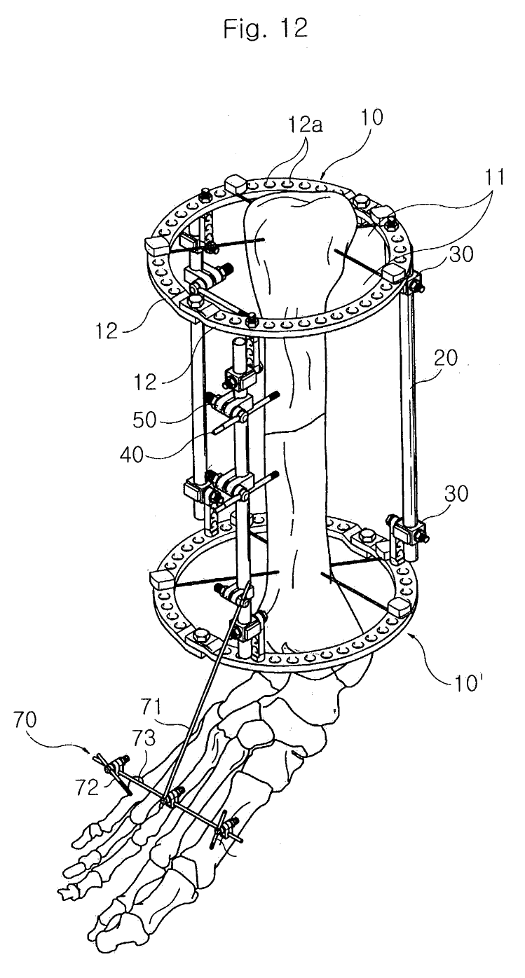

As shown in FIG. 12, an extracorporeal fixing device for a bone fracture according to a fourth embodiment of the present invention further includes an assistant extracorporeal fixing device for a bone fracture 70 which includes a first assistant rod 71 having one end movably fixed to the rod 20 by the clamp, and a second assistant rod 73 in which an assistant fixing pin 72 engaged to the other end of the first assistant rod 71 by a clamp and is adapted to fix the back of hand and the top of foot.

While the extracorporeal fixing device for a bone fracture according to the embodiments of the present invention is described referring to the attached figures as above, the present invention is not be to limited to only the specifically preferred embodiments and figures depicted herein and variations and modifications within the scope of the invention may occur. For example, the ring-rod fixing clamp according to the second embodiment of the present invention can be used as a rod-pin fixing clamp which engage fixing pin movably to the rod by modifying the diameter of the through hole. The fixing pin pass through the head portion of the rod fixing bolt instead of the rod and the rod pass through the through hole positioned in one side of connection member in stead of the ring fixing member. Like this way, each fastening clamp can be modified into the ring-rod fixing clamp or rod-pin fixing clamp.

Such modification, alternatives, and equivalents may be included in the spirit and scope of invention as herein disclosed.

The extracorporeal fixing device for a bone fracture, according to the embodiments of the present invention depicted above, can be easily installed depending on the condition of the fractured bone and destructed soft tissue without regional restriction, and allows multi-directional modification and transformation, easy addition and disassembly, and can assure the firm fixing strength of conventional Ilizarov fixator.

Also, the space needed for the treatment of trauma can be easily obtained so that the treatment of trauma become more effective.

In addition, continuous radiation is not required so that the risk of overexposure to radiation can be minimized. And the use of costly image intensifier is not required so that economical treatment can be made.

What is better, it allows tidy treated region and minimize behavior restriction of patient by operating optimal treatment using minimal components.

What is claimed is:

1. Extracorporeal fixing device for a bone fracture comprising:
A) a pair of ring members through which an object bone structure pass in a longitudinal direction of the object bone structure and are spaced apart by a certain distance;
B) a rod which is installed between each of said ring members and adapted to setting a position of each member;
C) a pair of ring-rod fixing clamps, wherein each of said ring-rod fixing clamps engaged on a certain position of each said ring member, and said ring-rod fixing clamps rotatively fix both ends of said rod with respect to each ring member;
D) a fixing pin which is movably engaged to said rod and fixes a fracture portion;
E) a rod-pin fixing clamp which movably fixes said fixing pin to said rod.

2. Extracorporeal fixing device for a bone fracture as recited in claim 1, wherein said ring-rod fixing clamp comprising:
A) a ring fixing member which has one side being detachably engaged to said ring members and the other side having more than at least one engaging hole;
B) a first rod fixing member in which a through hole which an end of said rod passes through formed in an middle portion, and threaded portions formed in an upper and lower portion wherein said lower threaded portion between said threaded portions passes through said engaging hole of said ring fixing member and is engaged by a nut;
C) a rod fixing piece which is implemented in such a manner that said upper threaded portion of said first rod fixing member passes through in an upper direction, and a front and back portion of said first rod fixing member is surrounded, and a through hole corresponding to the through hole of said first rod fixing member is formed in a front and back portion contacting with a front and back surface of said first rod fixing member, for thereby fixing said rod to said through hole of said first rod fixing member by downwardly pressurizing said rod based on a tightening force of said nut engaged to said upper threaded portion of said first rod fixing member.

3. Extracorporeal fixing device for a bone fracture as recited in claim 1, wherein said ring-rod fixing clamp comprising:
A) a ring fixing member which has one side being detachably engaged to said ring members;
B) a rod fixing bolt which has a head portion through which an end portion of said rod passes vertically;
C) a connection member in which a through hole through which the other end of said ring fixing member passes horizontally is formed in one side, and upper and lower corresponding surfaces which are extended from one side in the upper and lower directions are formed in the other side, and a through hole through which said rod fixing bolt passes vertically is formed in each said corresponding surface;
D) a rod fixing piece which is installed between a head portion of said rod fixing bolt and said connection member and fixes said rod to said rod fixing bolt by pressurizing an end portion of said rod based on a tightening force of a nut engaged to threaded portion of said rod fixing bolt which passes through said connection member, and forms occlusion with corresponding surface of said connection member so as to prevent a relative rotation.

4. Extracorporeal fixing device for a bone fracture as recited in claim 1, wherein said rod-pin fixing clamps comprising:
A) a pin fixing bolt which has a through hole through which said fixing pin passes vertically;
B) a second and a third rod fixing member, wherein one end of each of said second and third rod fixing member has a through hole which said pin fixing bolt passes through in sequence, and an insertion groove into which an end of said rod is inserted, is formed in the opposite other end of said second and third rod fixing member;
C) a pin fixing piece which is installed between a head portion of said pin fixing bolt and said second rod fixing member and fixes said fixing pin to said through hole of said pin fixing bolt by pressurizing said fixing pin based on a tightening force of a nut engaged to threaded portion of said pin fixing bolt, and forms an occlusion with corresponding surface of said second rod fixing member so as to prevent a relative rotation.

5. Extracorporeal fixing device for a bone fracture as recited in claim 4, further comprising a pair of escape prevention rod which has one end passing through a groove formed in both sides of through hole of said third rod fixing member and being integrally engaged to an opposite surface of said second rod fixing member, and the other end in which an engaging portion is formed for preventing an escape of said third rod fixing member in such a manner that said engaging portion is caught by a bottom of said groove formed in both sides of through hole of said third rod fixing member.

6. Extracorporeal fixing device for a bone fracture as recited in claim 4 or 5, wherein a rotation prevention groove is formed in the threaded portion of said pin fixing bolt in a longitudinal direction and a protrusion which is inserted into said rotation prevention groove is formed in an inner circumference of said through hole of said pin fixing piece, so that a relative rotation of said pin fixing piece is prevented with respect to said pin fixing bolt.

7. Extracorporeal fixing device for a bone fracture as recited in claim 4, wherein one side of through hole formed in said pin fixing bolt of said rod-pin fixing clamp is opened, so that said fixing pin is inserted through the opened one side or is escaped therefrom.

8. Extracorporeal fixing device for a bone fracture as recited in claim 1, further comprising an assistant extracorporeal fixing device for a bone fracture which is movably fixed to said rod and fixes the back of hand and the top of foot.

9. Extracorporeal fixing device for a bone fracture as recited in claim 1, wherein said rod is made of carbon steel.

10. Extracorporeal fixing device for a bone fracture as recited in claim 1, wherein said ring members are made of carbon steel.
